(19) United States
(12) Patent Application Publication
(10) Pub. No.: US 2007/0197985 A1
(43) Pub. Date: Aug. 23, 2007

(54) Collection bag adapted for use in an incontinence management system

(75) Inventor: Mark Miskie, Charlotte, NC (US)

Correspondence Address: SCHWARTZ LAW FIRM, P.C., 6100 FAIRVIEW ROAD, SUITE 11350, CHARLOTTE, NC, 28210, US

(73) Assignee: ARCUS MEDICAL, LLC, Gastonia, NC (US)

(21) Appl. No.: 10/593,368

(22) PCT Filed: Mar. 18, 2004
(86) PCT No.: PCT/US04/08066

§ 371 Date: Sep. 18, 2006

(51) Int. Cl. A61F 5/44

(52) U.S. Cl. 604/349; 604/350

(57) ABSTRACT

A collection bag is adapted for communicating with a male incontinence device designed to cover a portion of the penis. The collection bag has first and second opposing flexible side walls joined together and forming a fluid container for holding urine. A cylindrical bag neck is formed with the first and second side walls, and defines a mouth for receiving urine passed through the incontinence device and into the fluid container. The bag neck comprises a rigid valve cap. The valve cap includes an internal check valve for controlling urine flow outwardly from and into the incontinence device, and multiple circumferentially-spaced indexing elements. The indexing elements cooperate to position the valve cap relative to the incontinence device, such that the check valve is locatable in a single operative orientation.

### TECHNICAL FIELD AND BACKGROUND OF THE INVENTION

This invention relates generally to male incontinence, and more specifically to a novel urine collection bag adapted for use in an improved incontinence management system. The invention resides relatively unnoticed under clothing, and offers greater conveniences and an increased freedom of movement often sacrificed with commercially available male incontinence devices and catheters. Users can wear the invention with confidence and without a feeling of self-consciousness.

Incontinence is a growing problem, particularly in those adults ages 65 and older. Approximately four million males in the United States suffer from varying degrees of incontinence. Common causes for this condition include prostate cancer, pelvic trauma, spinal cord injury, medication side-effects, and resulting effects of certain medical conditions such as Alzheimer's and diabetes. Over $16 billion is spent annually on incontinence related care.

The present invention addresses several disadvantages and limitations of prior art collection bags used in certain existing devices and catheters for managing male incontinence. Specifically, the invention effectively eliminates urine back flow, is quickly and easily applied to the incontinence device, and is conveniently emptied when full. The invention is also applicable for monitoring the amount of urine expelled by patients in bladder training during rehabilitation. While not a cure for male urinary incontinence, the invention is intended to allow for a better quality of life with greater conveniences and fewer problems as compared to present, commercially available and accepted incontinent devices.

### SUMMARY OF INVENTION

Therefore, it is an object of the invention to provide a urine collection bag which is especially adapted for use in an incontinence management system.

It is another object of the invention to provide a urine collection bag which can be worn relatively unnoticed under clothing and without a feeling of self-consciousness.

It is another object of the invention to provide a urine collection bag which is especially designed for active male adults suffering from moderate to heavy urinary incontinence.

It is another object of the invention to provide a urine collection bag which is conveniently cleaned and reuseable.

It is another object of the invention to provide a urine collection bag which, according to an alternative embodiment, is disposable.

It is another object of the invention to provide a urine collection bag which is relatively inexpensive to manufacture.

It is another object of the invention to provide a urine collection bag which includes means for readily ascertaining the level of urine contained in the bag.

It is another object of the invention to provide a urine collection bag which includes a deodorizing agent.

It is another object of the invention to provide a urine collection bag which attaches to a male incontinence device in a single operative orientation.

It is another object of the invention to provide a urine collection bag which incorporates a permanently attached or removable rigid valve cap.

It is another object of the invention to provide a urine collection bag which effectively eliminates the problem of urine back flow.

It is another object of the invention to provide a novel valve cap which is adapted for use in an incontinence management system, or other related or unrelated applications.

These and other objects of the present invention are achieved in the preferred embodiments disclosed below by providing a collection bag adapted for communicating with a male incontinence device designed to cover a portion of the penis. The collection bag has first and second opposing flexible side walls joined together and forming a fluid container for holding urine. A cylindrical bag neck is formed with the first and second side walls, and defines a mouth for receiving urine passed through the incontinence device and into the fluid container. The bag neck comprises a rigid valve cap. The valve cap includes an internal check valve for controlling urine flow outwardly from and into the incontinence device, and multiple circumferentially-spaced indexing elements. The indexing elements cooperate to position the valve cap relative to the incontinence device, such that the check valve is locatable in a single operative orientation. The valve cap may be integrally-formed with bag neck, or formed as a separate and replaceable component.

According to another preferred embodiment of the invention, the valve cap has an enlarged connecting portion adapted for receiving an open end of the incontinence device, and an integrally-formed cylindrical valve housing containing the check valve.

According to another preferred embodiment of the invention, the indexing elements are spaced-apart along an inner circumference of the connecting portion of the valve cap.

According to another preferred embodiment of the invention, an outer circumference of the connecting portion has visual markers adapted to facilitate proper application of the rigid valve cap to the incontinence device.

According to another preferred embodiment of the invention, a perforated inlet wall is formed within the valve housing.

According to another preferred embodiment of the invention, the check valve is a pivoted disk which resides in a normally closed position seated against the perforated inlet wall to restrict back flow of fluid from the fluid container to the incontinence device. When under a minimum crack pressure, the pivoted disk moves to a temporarily open position away from the perforated inlet wall to allow free fluid flow from the incontinence device to the fluid container.

According to another preferred embodiment of the invention, a retainer ring resides inside the valve housing, and is adapted for holding the pivoted disk in position adjacent the perforated inlet wall.

According to another preferred embodiment of the invention, the pivoted disk is formed of a flexible elastomer.

According to another preferred embodiment of the invention, at least one of the opposing side walls is at least semi-transparent.

In another embodiment, the invention is an incontinence management system including an elongated receptacle designed to cover a portion of the penis, and a collection bag communicating with the receptacle. The collection bag includes first and second opposing flexible side walls joined together and forming a fluid container for holding urine. A cylindrical bag neck is formed with the first and second side walls and defines a mouth for receiving urine passed through the incontinence device and into the fluid container. The bag neck comprises a rigid valve cap. The valve cap includes an internal check valve for controlling urine flow outwardly from and into the incontinence device, and multiple circumferentially-spaced indexing elements. The indexing elements cooperate to position the valve cap relative to the incontinence device, such that the check valve is locatable in a single operative orientation.

In yet another embodiment, the invention is a rigid valve cap adapted for use in an incontinence device. The valve cap includes an internal check valve for controlling urine flow outwardly from and into the incontinence device, and multiple circumferentially-spaced indexing elements. The indexing elements cooperate to position the valve cap relative to the incontinence device, such that the check valve is locatable in a single operative orientation. The valve cap may be integrally-formed with the incontinence device, or formed as a separate and replaceable component which communicates with the incontinence device.

### BRIEF DESCRIPTION OF THE DRAWINGS

Some of the objects of the invention have been set forth above. Other objects and advantages of the invention will appear as the description proceeds when taken in conjunction with the following drawings, in which:

FIG. 1 is a perspective view of the collection bag according to one preferred embodiment of the present invention, and showing the bag disconnected from the receptacle;

FIG. 2 is an exploded perspective view of the rigid valve cap;

FIG. 3 is a top view of the valve cap;

FIG. 4 is a fragmentary view of the collection bag and receptacle, and showing the receptacle neck properly oriented prior to application to the valve cap;

FIG. 5 is a fragmentary view of the collection bag and receptacle, and showing the receptacle neck partially inserted into the connecting portion of the valve cap;

FIG. 6 is a cross-sectional view of the receptacle neck positioned within the connecting portion of the valve cap;

FIG. 7 is a cross-sectional view of the receptacle neck positioned within the connecting portion of the valve cap, and rotated into a locked position to operatively interconnect the bag and receptacle;

FIG. 8 is a cross-sectional view of the valve cap with the receptacle neck received within the connecting portion and valve housing, and showing the locking arrow and tab (in phantom) in an engaged locking condition;

FIG. 9 is a cross-sectional view of the valve cap with the receptacle neck removed, and showing the location of the check valve in the normally closed position adjacent the perforated inlet wall; and

FIG. 10 is a cross-sectional view of the valve cap with the receptacle neck removed, and demonstrating operation of the check valve in the open position pivoted away from the perforated inlet wall under a minimum crack pressure.

### DESCRIPTION OF THE PREFERRED EMBODIMENT AND BEST MODE

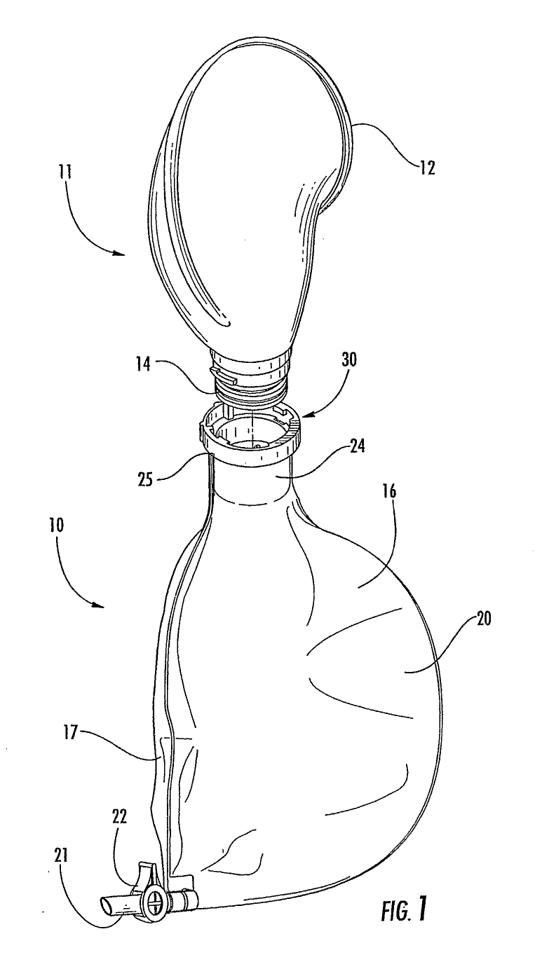

Referring now specifically to the drawings, a urine collection bag according to the present invention is illustrated in FIG. 1, and shown generally at reference numeral 10. The collection bag 10 is especially applicable for use in an incontinence management system including an ergonomically-designed, shape-retaining male receptacle 11. The receptacle 11 has a relatively large diameter open end 12 adapted to receive the penis, and an opposite end defining an elongated reduced-diameter neck 14 communicating with the collection bag 10, as described below. When in use, urine leakage passes outwardly through the receptacle 11 and into the collection bag 10 where the urine is temporarily stored prior to disposal. Both the collection bag 10 and receptacle 11 may be conveniently carried in a supporting undergarment (not shown) having suitable pockets and other accommodating structure.

The collection bag 10 is constructed of first and second opposing flexible side walls 16 and 17 joined together and forming a fluid container 20 for holding urine. Preferably, at least one of the side walls 16, 17 is formed of a semi-transparent, white or skin tone polymer which allows the user to visually determine the level of captured urine contained in the fluid container 20. According to one embodiment, the thickness of each side wall 16, 17 is at least 8 mil. The capacity of the fluid container 20 is approximately 500 ml (cc). When the fluid container 20 is full, urine is conveniently discharged from the collection bag 10 through a horizontal drain tube 21 including a user-actuated flow control valve 22.

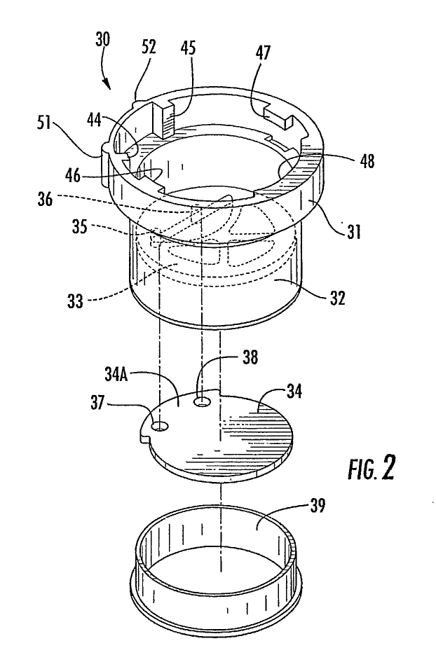

As best shown in FIGS. 1 and 2, the collection bag 10 has a generally cylindrical neck 24 integrally formed with the first and second side walls 16, 17 of the container 20, and defining a mouth 25 for receiving urine passing through the receptacle 11. In one embodiment, the bag neck 24 comprises a separately molded, rigid valve cap 30. The valve cap 30 is located at the mouth 25 of the container 20, and is permanently attached to an interior of the container walls 16, 17 by means, such as gluing or heat welding. The valve cap 30 has an enlarged-diameter connecting portion 31 adapted for receiving the receptacle neck 14, and an integrally-molded, reduced-diameter, cylindrical valve housing 32 extending inside the container 20 and permanently attached to the container walls 16, 17. A perforated inlet wall 33 is formed within the valve housing 32, and cooperates with a pivoted check valve 34 to control one-way flow of urine outwardly from the receptacle 11 and into the bag 10. Preferably, the check valve 34 comprises a thin, flexible, resilient, silicone rubber disk. The check valve 34 is carried on a pair of support posts 35 and 36 projecting from the inlet wall 33 and extending through complementary holes 37 and 38 formed in an integral hinge portion 34A of the check valve 34. The hinge portion 34A is sandwiched between the inlet wall 33 and a retaining ring 39 permanently glued to an interior of the valve housing 32. The retaining ring 39 engages the hinge portion 34A and holds the check valve 34 in a normally closed position directly adjacent the perforated inlet wall 33. When under a minimum crack pressure, the check valve 34 pivots to an open position, as described further below.

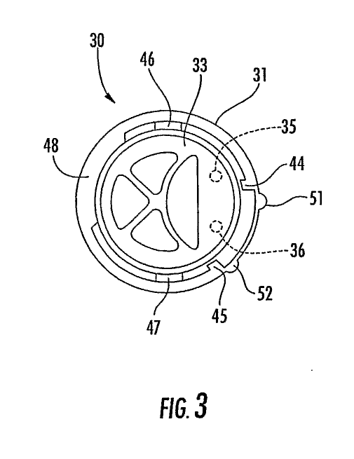

The perforations formed with the inlet wall 33 are strategically designed and arranged, as best shown in FIG. 3, and cooperate with the check valve 34 to promote efficient and effective one-way urine flow outwardly from the receptacle 11. In order to maintain proper operation of the check valve 34, the collection bag 10 and receptacle 11 are designed to interconnect in a manner such that the check valve 34 locates in a single operative orientation relative to the receptacle 11. This orientation is dictated by cooperating indexing elements formed with the receptacle neck 14 and the enlarged-diameter connecting portion 31 of the valve cap 30.

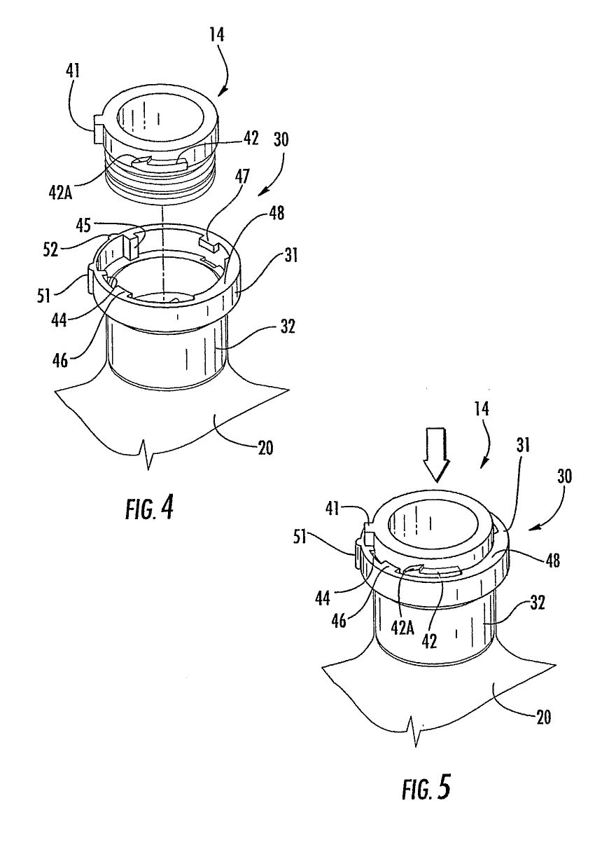

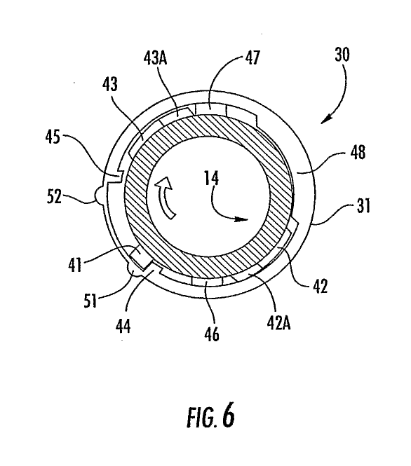

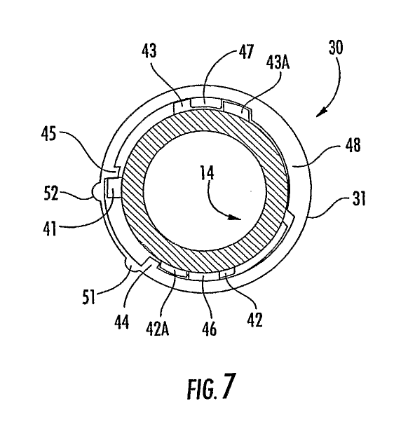

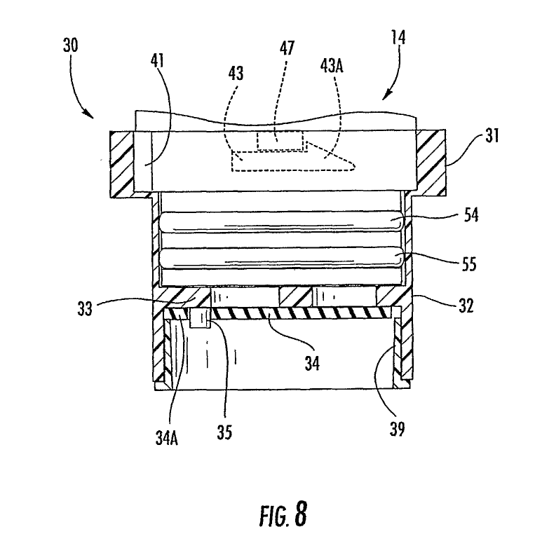

FIGS. 4-8 demonstrate proper application of the receptacle 11 to the collection bag 10. The receptacle neck 14 includes an indexing finger 41 and opposing circumferentially-spaced locking arrows 42 and 43. The connecting portion 31 of the bag's valve cap 30 has circumferentially-spaced pivot stops 44 and 45, locking tabs 46 and 47, and a raised interior wall 48. As best shown in FIGS. 5 and 6, prior to inserting the receptacle neck 14 into the valve cap 30, the indexing finger 41 is first aligned with a visual marker 51 formed with an outer circumference of the connecting portion 31. This alignment locates the locking arrow 42 between the locking tab 46 and raised wall 48, and the locking arrow 43 between the pivot stop 45 and locking tab 47 (See FIG. 6). Once fully inserted, the receptacle neck 14 is rotated approximately 30 degrees, thereby moving the indexing finger 41 from pivot stop 44 to pivot stop 45. As shown in FIG. 7, at pivot stop 45, the indexing finger 41 aligns with a second visual marker 52 formed with the outer circumference of the connection portion 31. In this position, respective heads 42A, 43A of the locking arrows 42, 43 pass under the locking tabs 46, 47 (See FIG. 8) to snap-attach the receptacle neck 14 to the bag's valve cap 30. In this resulting orientation, the check valve 34 is designed to open outwardly from the lowest point of urine collection within the neck 14 of the receptacle 11. The circumferential spacing of the locking arrows 42, 43 and tabs 46, 47 further promotes relatively uniform distribution of force interconnecting the bag 10 and receptacle 11. Preferably, the connection is further sealed by rubber O-rings 54 and 55 (See FIG. 8) located at the end of the receptacle neck 14 and engaging an interior wall of the valve housing 32. The receptacle 11 is disconnected from the bag 10 by pressing the receptacle neck 14 and valve cap 30 together to frictionally disengage the locking arrows 42, 43 and tabs 46, 47, and then twisting the valve cap 30 to reposition the indexing finger 41 adjacent the pivot stop 44.

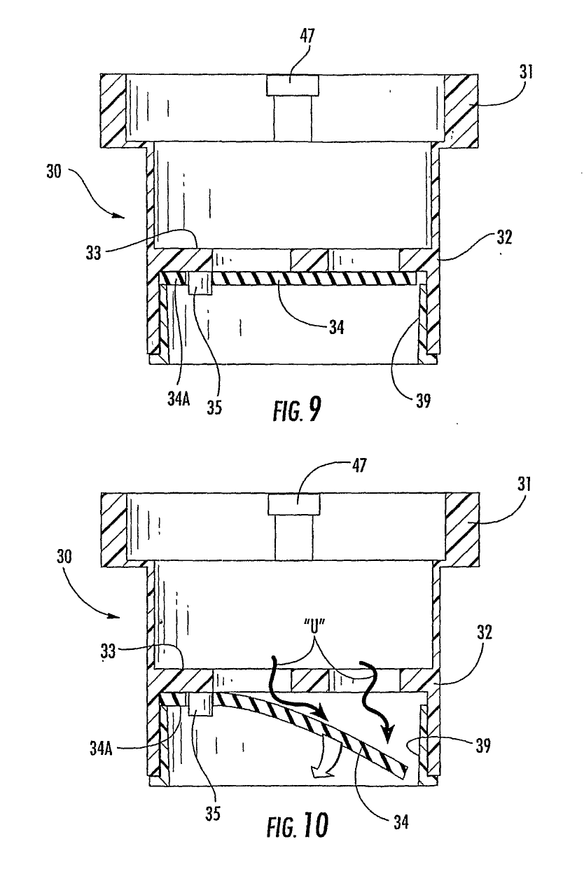

FIGS. 9 and 10 demonstrate operation of the check valve 34. In a normal condition, the check valve 34 remains closed, as shown in FIG. 9, and prevents any back flow of urine from the fluid container 20 into the receptacle 11. When leakage occurs, as indicated by direction arrows “U”, the check valve 34 temporarily opens away from the perforated inlet wall 33 pivoting adjacent the fixed hinge portion 34A to enable urine flow outwardly from the receptacle 11 and into the container 20. When urine flow ceases, the inherent biasing force created by the integral hinge portion 34A causes the check valve 34 to promptly return to its normal, closed position seated against the perforated inlet wall 33. Preferably, the check valve 34 has a crack pressure of approximately 0.02 psi at one-half cubic inch of fluid head, and a burst (back flow) pressure of at least 20 psi. The maximum flow rate of urine through the check valve 34 and into the container 20 is at least 30 cc/sec (1 oz/sec). For convenient attachment and detachment, the collection bag 10 may have touch fastener tabs (not shown) adapted to mate with complementary fasteners pads located on the supporting undergarment.

A urine collection bag for a male incontinence device is described above. Various details of the invention may be changed without departing from its scope. Furthermore, the foregoing description of the preferred embodiment of the invention and best mode for practicing the invention are provided for the purpose of illustration only and not for the purpose of limitation—the invention being defined by the claims.

I claim:

1. A collection bag adapted for communicating with a male incontinence device designed to cover a portion of the penis, said collection bag comprising:
(a) first and second opposing flexible side walls joined together and forming a fluid container for holding urine; and
(b) a cylindrical bag neck formed with said first and second side walls and defining a mouth for receiving urine passed through the incontinence device and into said fluid container, said bag neck comprising a rigid valve cap, and said valve cap comprising:
(i) an internal check valve for controlling urine flow outwardly from and into the incontinence device;
(ii) multiple circumferentially-spaced indexing elements; and
(iii) said indexing elements cooperating to position said valve cap relative to the incontinence device, such that said check valve is locatable in a single operative orientation.

2. A collection bag according to claim 1, wherein said valve cap comprises an enlarged-diameter connecting portion adapted for receiving an open end of the incontinence device, and an integrally-formed, reduced-diameter cylindrical valve housing containing said check valve.

3. A collection bag according to claim 2, wherein said indexing elements are spaced-apart along an inner circumference of the connecting portion of said valve cap.

4. A collection bag according to claim 2, wherein an outer circumference of said connecting portion comprises visual markers adapted to facilitate application of said rigid valve cap to the incontinence device.

5. A collection bag according to claim 2, and comprising a perforated inlet wall formed within said valve housing.

6. A collection bag according to claim 5, wherein said check valve comprises a pivoted disk residing in a normally closed position seated against said perforated inlet wall to restrict back flow of fluid from said fluid container to the incontinence device, and when under a minimum crack pressure, said pivoted disk moving to a temporarily open position away from the perforated inlet wall to allow free fluid flow from the incontinence device to said fluid container.

7. A collection bag according to claim 6, and comprising a retainer ring residing inside said valve housing and adapted for holding said pivoted disk in position adjacent the perforated inlet wall.

8. A collection bag according to claim 6, wherein said pivoted disk is formed of a flexible elastomer.

9. A collection bag according to claim 1, wherein at least one of said opposing side walls is at least semi-transparent.

10. An incontinence management system, comprising:
(a) an elongated receptacle designed to cover a portion of the penis; and
(b) a collection bag communicating with said receptacle, and comprising:
(i) first and second opposing flexible side walls joined together and forming a fluid container for holding urine; and
(ii) a cylindrical bag neck formed with said first and second side walls and defining a mouth for receiving urine passed through the incontinence device and into said fluid container, said bag neck comprising a rigid valve cap, and said valve cap comprising:
an internal check valve for controlling urine flow outwardly from and into the incontinence device;
multiple circumferentially-spaced indexing elements; and
said indexing elements cooperating to position said valve cap relative to the incontinence device, such that said check valve is locatable in a single operative orientation.

11. An incontinence management system according to claim 10, wherein said receptacle comprises an outer shape-retaining shell having a reduced-diameter receptacle neck defining an open end communicating with the mouth of said bag neck.

12. An incontinence management system according to claim 11, wherein said valve cap comprises an enlarged-diameter connecting portion receiving the open end of said receptacle neck, and an integrally-formed, reduced-diameter cylindrical valve housing containing said check valve.

13. An incontinence management system according to claim 12, wherein said receptacle neck comprises complementary indexing elements cooperating with the indexing elements of said valve cap to locate said check valve in its single operative orientation.

14. An incontinence management system according to claim 13, wherein the indexing elements of said valve cap comprise first and second fingers extending radially inward from an inner circumference of said connecting portion, and wherein the indexing elements of said receptacle neck comprise an outwardly extending finger cooperating with the fingers of said valve cap to limit rotation of said valve cap relative to the receptacle.

15. An incontinence management system according to claim 14, wherein the indexing elements of said valve cap further comprise locking tabs.

16. An incontinence management system according to claim 15, wherein the indexing elements of said receptacle neck further comprise complementary locking arrows frictionally engaging the locking tabs of said valve cap in a locked condition.

17. A rigid valve cap adapted for use in an incontinence device, said valve cap comprising:
(a) an internal check valve for controlling urine flow outwardly from and into the incontinence device;
(b) multiple circumferentially-spaced indexing elements; and
(c) said indexing elements cooperating to position said valve cap relative to the incontinence device, such that said check valve is locatable in a single operative orientation.

18. A valve cap according to claim 17, wherein said valve cap comprises an enlarged-diameter connecting portion adapted for receiving an open end of the incontinence device, and an integrally-formed, reduced-diameter cylindrical valve housing containing said check valve.

19. A valve cap according to claim 18, and comprising a perforated inlet wall formed within said valve housing.

20. A valve cap according to claim 19, wherein said check valve comprises a pivoted disk residing in a normally closed position seated against said perforated inlet wall to restrict back flow of fluid into the incontinence device, and when under a minimum crack pressure, said pivoted disk moving to a temporarily open position away from the perforated inlet wall to allow free fluid flow outwardly from the incontinence device.
